(19) United States
(12) Patent Application Publication
(10) Pub. No.: US 2013/0266536 A1
(43) Pub. Date: Oct. 10, 2013

(54) METHOD OF USE OF STABILIZED PLANT-DERIVED GROWTH FACTOR IN SKIN CARE

(71) Applicant: Ágústa Guõmundsdóttir, Reykjavik (IS)

(72) Inventors: Einar Mantyla, Reykjavik (IS); Bjorn Larus Orvar, Kopavogur (IS); Jon Bragi Bjarnason, Reykjavik (IS); Yves Balmer, Sion (CH)

(21) Appl. No.: 13/520,964

(22) PCT Filed: Jan. 6, 2011
(86) PCT No.: PCT/IS11/50001

§ 371 Date: Apr. 15, 2013

(30) Foreign Application Priority Data
Jan. 6, 2010 (IS) 8871

(51) Int. Cl. A61K 38/20; A61K 38/18

(52) U.S. Cl. CPC A61K 38/2006; A61K 38/1808; A61K 38/1866; A61K 38/18; A61K 38/1841
USPC 424/85.2; 514/9.6; 514/8.1; 514/8.6; 514/8.9

(57) ABSTRACT

Cosmetic and dermatologic compositions for skin care, containing a transgenic plant extract containing a growth factor, or a growth factor purified from transgenic plants, or a mixture of growth factors derived from transgenic plants as extracts or in purified form, for use in topical therapeutics, dermatology and cosmetics. Importantly this invention provides stabilized, safer growth factors available for use for cosmetic and topical treatment. Preferred composition comprises a plant-produced growth factor and hyaluronic acid. The skin-care/dermatological compositions with stabilized growth factor do not carry the risk of unwanted breakdown products and the resulting loss of activity of the composition. Furthermore, the composition is without contaminants and transmissible agents that can result from animals or animal or bacterial cell based expression systems.

### FIELD OF THE INVENTION

The present invention generally relates to cosmetic and dermatological compositions comprising stabilized growth factors and cytokines for skin care, dermatological applications and methods for making skin care products. In particular, this invention relates to stabilized heterologous growth factors obtained from transgenic plants and their use in cosmetic and pharmaceutical products.

### BACKGROUND OF THE INVENTION

Skin is the biggest organ of the human body carrying out various functions such as protection, barrier, temperature controlling, excretion and respiration. With time and ageing, those functions rapidly decline, and a variety of physiological changes occur to the skin. These changes are manifested in the decrease in the thickness of epidermis, dermis and subcutaneous tissue, which are the main components of skin. Changes in lipid composition undermine the moisture barrier role of lipid layers and resulting in the dryness of skin. Further, with age, the occurrence of age spots, freckles, pigmentation or various skin lesions also increases.

An age-dependent decrease in epidermal turnover rate is involved in accumulation of low quality Stratum Corneum, resulting in senile xerosis, undue pigmentation and fine wrinkles. This may, in part, be due to aberrant keratinocyte differentiation.

Environmental components such as pollution and UV-rays, can speed up the ageing of the skin. Reactive oxygen species and free radicals and some physiological states such as fatigue or stress are particularly detrimental to proteins, nucleic acids and membrane lipids, leading to the aging of the skin. Accordingly, there have been many studies on the occurrence of the wrinkles, age spots or freckles, the loss of skin elasticity, the pigmentation, and the dryness and cracking of skin.

A variety of cosmetic compositions have been developed in order to prevent or slow down the problems of aging of the skin and skin wrinkles with the aim of improving wrinkles, sagging and the reduction in elasticity of skin caused by sunlight. Japanese Patent Laid-open Publication No. Hei 5-246838 discloses a method for improving wrinkles of skin by the synthesis of collagen. It teaches that the activity of collagenase that decomposes collagen to promote collagen metabolism might be reduced with aging, leading to the increase of cross-link collagens and the increase of skin wrinkles.

### Skin Conditions:

Psoriasis is a skin condition resulting in red coloration of the skin and scaly patches and peeling of skin at the spot. Psoriatic patches are sites of excess skin production due to changes in cell development and rate of cell division in the epidermis resulting from altered growth behavior of these cells. It is known that cells at the site of psoriatic patch produce cell signaling compounds resulting in inflammatory response. It has been hypothesized that psoriasis is a form of immune response that may result in self-incompatibility that can be triggered by external conditions such as infections.

### Eczema:

Eczema is a form of dermatitis, or inflammation of the epidermis. The term eczema is broadly applied to a range of persistent skin conditions. These include dryness and recurring rashes that can have one or more of the symptoms redness, skin edema (swelling), itching and dryness, crusting, flaking, blistering, cracking, or even oozing or bleeding. Dermatitis is often treated with corticosteroids. Because of the risks associated with corticoids such as thinning of skin, steroids must be sparingly applied only to control an episode of eczema.

When eczema is severe and does not respond to other forms of treatment, immunosuppressant drugs such as cyclosporin are sometimes prescribed. These dampen the immune system and can improve the eczema, but can cause side effects.

For severe itching, sedative antihistamines can be used but may cause drowsiness.

Scar tissue is a mark left on damaged tissue both internally and externally such as on skin after it has healed after surgery or wounding. Scar tissue is dense fibrous connective tissue that forms over and/or around a healed wound or cut, and can affect negatively the elasticity of the skin and cause esthetic problems when visible on the skin and inconvenience. Extensive scar tissue can have negative effects on appearance and quality of life of individuals recovering from traumatic experiences such as burns.

Growth factors are key players in regulating proliferation and differentiation of cells and are involved in restructuring the epidermis and basal lamina upon injury or damage. They are important for the renewal of cells and thus, can counteract several aspects of aging and normalize keratinocyte differentiation, fibroblast growth and induce turnover and renewal of cells and cellular products.

Ito et al. (2009) teach that the expression of Matrix metalloproteinases (MMPs) and tissue inhibitors of metalloproteinase (TIMPs) in airway smooth muscle (ASM) cells could be involved in collagen turnover and migration of these cells and thus may contribute to airway remodeling. PDGF strongly up-regulates the expression of Matrix Metalloprotease-1 (MMP-1) at mRNA and protein levels. PDGF, when combined with TGF-beta, caused synergistic up-regulation of MMP-3. TIMP-1 was additively up-regulated by TGF-beta and PDGF.

Nakatani et al. (2009) teach that hyaluronan affects expression and protein levels of MMP-1 in periodontal ligament cells. Hyaluronan oligo (HAoligo) remarkably enhanced MMP-1 expression in both mRNA and protein levels, but no effect was shown on the expression of TIMP-1 and TIMP-2 mRNAs. It was suggested that HAoligo induces MMP-1 expression in HPDL cells, and p38MAPK plays a crucial role in signal transduction for MMP-1 inducted by HAoligo.

Although the examples of Ito and Nakatani are limited to signal pathways in airway smooth muscle and ligament cells they describe separately how growth factors and hyaluronan may affect the restructuring and turnover of components of extracellular matrix and basal lamina.

It is recognized that growth factors can have beneficial effects on various skin disorders and skin injuries and counteract effects of aging that are the result of impaired or deteriorating protective mechanisms at cellular level. Growth factors can promote cellular renewal and proliferation and are a natural component of the healing process of wounds.

The Epidermal Growth Factor (EGF) promotes the division of various epithelial cells originated in the ectoderm and mesoderm. It is extensively distributed in body fluid, especially in urine and breast milk (Carpenter, G. and Cohen, S., “Epidermal growth factor,” Ann Rev. Biochem., 48, 192-216 (1979)). It is a single polypeptide consisting of 53 amino acid residues and has a molecular weight of 6,200 Daltons (Campion, S. R. and Niyogi, S. K., “Interaction of epidermal growth factor with its receptor”). In 1962, Cohen isolated EGF from the gland beneath the chin of the mature male mouse. In 1972, Savage and Taylor identified the primary structure of mouse EGF and the location of three intramolecular disulfide bonds in EGF that are essential for physiological function.

EGF is believed to have an excellent effect on skin injuries because it strongly promotes the proliferation of epithelial cells, endothelial cells and fibroblasts, and also the migration and proliferation of epithelial cells to where they are deficient. Growth factors are key players in maintenance of tissue integrity and in cell to cell communication, thus playing a protective role in fighting degeneration of epidermal tissue.

U.S. Pat. No. 5,618,544 incorporated herein by reference in its entirety, discloses a cosmetic composition comprising EGF, TGF-a and FGF for decreasing cutaneous senescence and improving the appearance of skin.

U.S. Pat. No. 6,589,540 teaches that EGF remarkably enhances the effect of retinol used in cosmetics, and also effectively alleviates the skin irritation of retinol.

Growth factors such as PDGF are released at wound site during coagulation phase, and act as chemo-attractants for neutrophils, macrophages and fibroblasts. These cells play an important role in killing bacteria and removal of necrotic debris at the wound site. Activated macrophages release in turn growth factors that promote angiogenesis and communicate with the B-cell and T-cell mediated immune responses. Macrophages secrete TGF-beta, that stimulates fibroblasts to produce new extracellular matrix, and VEGFs that stimulate angiogenesis. Epithelization proceeds as keratinocytes divide and cover the wound bed. Thus, it is well established that growth factors are important mediators of healing process and studies indicate that G-CSF may be beneficial for treating infected diabetic ulcerations. EGF stimulates the proliferation of fibroblasts and keratinocytes.

FGF has proliferative effects on epithelial cells and has been observed to accelerate bone and wound healing in animal models. KGF −2 accelerates wound healing significantly, especially the closing of wounds.

Growth factors isolated from animal tissue or blood carry the risk of unwanted contaminants and transmissible agents, such as but not limited to viruses, virions, prions, other co-purifying growth factors. The same risk of contaminating transmissible agents and endogenous growth factors is present in growth factors produced in animal or human cells by biotechnological means. Growth factors produced with biotechnological means in bacteria pose the risk of carry-over of bacterial endotoxins that are known to disturb the immune system. Frequently, bacteria are unable to produce growth factors or cytokines in their native form, but deform their tertiary structure, necessary for their bioactivity, to a denatured state and pack them into inclusion bodies within the cells. The denatured growth factors from isolated inclusion bodies require a challenging and extensive refolding in order to regain bioactivity. Furthermore, bacteria are unable to glycosylate proteins, which in several cases are known to make them less stable and more prone to degradation by proteases. The risk of transmissible agents, endotoxins or contaminants is clearly of concern for the use of growth factors produced in bacteria, yeast- or animal cells to treat open wounds and challenged epidermis with symptoms of inflammation, and thus use in dermatological and cosmetic applications.

There is continued demand for growth factors and other biologically active proteins of high quality, prepared such as to minimize or eliminate the above problems and disadvantages.

### SUMMARY OF THE INVENTION

Growth factors and cytokines produced in plants are free from transmissible infectious agents such as animal or human viruses, virions and prions and bacterial endotoxins. There are no reported cases of plant diseases that could cause diseases in man, in contrary to numerous animal diseases that can infect man. Thus, plants constitute a safer production organism than the above mentioned cell types, animal cells, animal tissues, yeast and bacteria, for the production of growth factors. Plants lack an immune system comparable to that of animals that requires the action and participation of growth factors as signaling elements. Plants therefore do not produce themselves growth factors similar to animal or human growth factors, providing a pristine host source for recombinant growth factors using transgenic technology. Plants are able to glycosylate proteins, which improves their stability and can affect their activity, and therefore are able to produce superior growth factors compared to those produced in bacteria. Plants produce growth factors and cytokines in their native bioactive form that are, thus, of superior quality than denatured and refolded growth factors produced in bacteria. Production of growth factors in plants with biotechnological means according to the invention circumvents these safety, quality, and purity problems. Plant-derived growth factors whether in extract or in purified native form are therefore safer and cleaner for use in dermatology, topical therapeutics, skin grafts, hair transplants, skin care and cosmetics, than growth factors produced with current production methods.

Plants produce a number of proteins that play a protective role in the plant and alleviate stress caused by abiotic and biotic factors, such as dehydration and oxidative stress. Several of these proteins accumulate specifically in the seeds of a plant upon seed maturation that involves dehydration of the cellular tissue. Dehydrins are a class of proteins that accumulate in response to stress such as drought or as a part of a maturing process such as seed development.

It is an object of this invention to present methods of use of stabilized heterologous plant-produced recombinant growth factor in a hypoallergenic formulation enabling the topical use of growth factors in cosmetic and skin care products. The stability of the growth factor over extended time is crucial to its use as a topical treatment, as proteins are by nature sensitive to breakdown and catalysis.

It is an important feature of the present invention to present a formulation of heterologous plant-produced growth factor in a stabilizing composition with minimal number of ingredients, to obtain a non-allergenic, non-irritating composition that allows for the application and use of growth factors, not only to healthy skin but also to sensitive and challenged skin as in eczema and psoriasis.

It is an aspect of the invention to provide compositions with plant derived growth factor with positive mitigating effects on scar tissue formation during and after the healing of a wound, burn, pustule, ulcer, lesion or surgery, such as medical surgery and plastic surgery. This embodiment of the invention is particularly useful in reducing the signs of scar tissue on skin, thus improving the complexion and healing of ruptured skin, improving the appearance of the skin.

It is an object of the present invention to provide a cosmetic composition for skin care, containing a transgenic plant extract containing a growth factor, or a growth factor purified from transgenic plants, or a mixture of growth factors derived from transgenic plants as extracts or in purified form, for use in topical therapeutics and cosmetics. Plant extracts refer herein to protein extracts of the host plants (e.g. seed protein extracts) producing the heterologous growth factor of interest. The extract can be partially or substantially purified, indicating that the extract is enriched for the protein of interest with one or more suitable purification steps. Importantly, this invention makes safer growth factors available for use for cosmetic and topical treatment. These plant-produced growth factors may be glycosylated in plants when they carry glycosylation sites in their amino acid backbone, a feature that is known to improve stability of proteins and may affect their biological activity. Production of these active ingredients in their native form for compositions for cosmetic and topical use is made more economical by the present invention. More specifically, it is an object of the present invention to provide a skin-care composition comprising growth factor and optionally in a composition with other, naturally occurring, plant-based beneficial polypeptides, such as dehydrins and globulins in the extract. These seed proteins have a protective function at the cellular and biochemical level in plants and in the unique combination with a growth factor, as an object of this invention, they can provide nurturing and healing conditions and alleviate dehydration and oxidative stress at cellular level. It is an object of the present invention to provide a stable skin care composition suitable as the treatment of acne, inflammation of the epidermis, eczema, psoriasis, the improvement of skin wrinkles, age spots, freckles, blotches or other pigmentation, cracked epidermis particularly on hands, elbows, heels and feet, and the moisturizing of skin and wound healing and reduction of scar tissue.

In an aspect of the present invention, stabilized growth factors can be used to improve success of hair transplants by treatment of excised follicle units (FU) during and after the transplantation surgery.

A suitable growth factor or combination of two or more growth factors for the invention may be selected from plant-derived recombinant growth factors including but not limited to Epidermal growth factor (EGF), Keratinocyte growth factor (KGF), Platelet-derived growth factor (PDGF), Transforming growth factor-beta (TGF-beta), Tumor necrosis factor alpha (TNF-alpha), Vascular epidermal growth factor (VEGF), Neural growth factor (NGF), Insulin-like growth factors (IGFs), Fibroblast growth factor 2 (FGF-2), FGF acidic, Granulocyte Macrophage-Colony stimulating factor: GM-CSF, Granulocyte-Colony stimulating factor (G-CSF) Interleukins (IL-s, IL-1, ILl-alpha, IL-6, IL-8, IL-10), noggin, thymosin beta 4, and Bone morphogenesis proteins (BMPs). These plant-produced growth factors may be used according to the invention in healing of inflicted, pathological and surgical wounds and reduction/prevention of scar tissue formation. A selection of growth factors may be used for an ex vivo treatment in an operation such as hair-transplant, e.g. by immersing the excised follicle units in a solution containing plant-derived recombinant growth factors to improve viability of the excised follicle units and to speed up and progress the healing process following the transplantation.

A choice of growth factors such as thymosin beta 4 and noggin are examples of preferred growth factors for this use of the invention, they are found to disrupt a refractive stage of cells in hair follicules at post-transplantational stage, and induce hair growth. The present invention provides compositions and means to treat scalp and follicles and/or follicle units (FU) in refractive stage with safe plant-derived human growth factors, in a hypoallergenic formula to revitalize hair growth and for healing from the effects of the transplantation surgery. It is a significant improvement in a number of therapeutic applications to be able to use recombinant human growth factors that are not sourced from human or animal source, and are not contaminated with bacterial endotoxins.

It is a further aspect of the invention to provide compositions with one or more plant-derived growth factor such as any of those above-mentioned and hyaluronan. Together, the effects of growth factors and hyaluronan on the metabolism of the skin can result in a positive synergistic effect on skin composition, normalizing cell differentiation, invigorating cell division and lead to renewal of components of the basal lamina, resulting in rejuvenated skin, wound healing and alleviation of persistent skin conditions and reduce inflammation.

In another aspect, the present invention provides a method of manufacturing a topical cosmetic product comprising providing a transgenic plant extract comprising a growth factor in a stabilizing medium. The plant-derived heterologous growth factor is preferably selected from any of the growth factors listed herein below. Preferably, the transgenic plant extract is a barley seed extract. The produced growth factors are in particular useful for making a skin-care/dermatological composition.

In yet a further aspect, the present invention provides one or more heterologous growth factor isolated from transgenic plants. The growth factors may also be used in other applications known to a skilled person in the art.

In a further aspect of the present invention, novel plant extracts containing growth factors are provided to be used for cosmetic purposes and as an active ingredient as in healing ointments or other forms of topical pharmaceutical compositions.

### BRIEF DESCRIPTION OF FIGURES

FIG. 1 shows two stained gels with transgenic plant extract containing EGF.

FIG. 2 shows two stained gels with transgenic plant extract containing VEGF.

FIG. 3 shows two stained gels with transgenic plant extract containing IGF-1

FIG. 4 shows two stained gels with transgenic plant extract containing IL-1a.

FIG. 5 shows a stability challenge test of a purified, reconstituted freeze-dried plant-made EGF.

FIG. 6 shows a subject hand exhibiting signs of winter eczema (a) before and (b) after application of a topical composition of the invention, as explained in Example 7.

FIG. 7 shows feet with “winter feet” (winter eczema) skin problems, (a) and (b) are pictures of the feet before and figures (c) and (d) after application for 5 days of topical composition of invention, as explained in Example 8.

FIG. 8: A typical skin deformation curve with measured and calculated parameters.

FIG. 9: Results of comparative measurement of facial skin mechanical properties treated with stabilized heterologous plant-derived EGF formulation and non-EGF control, in a split-face study, see Example 9.

FIG. 10: Results of non-invasive skin analysis measuring relative elasticity in 12 individuals receiving topical treatment with formulated plant extract containing plant-derived heterologous EGF (pd-EGF) or placebo.

### DETAILED DESCRIPTION OF THE INVENTION

As used herein, a “plant-derived” growth factor is a growth factor obtained from a transgenic plant or progenies of a transgenic plant and is used interchangeably with the term “plant-produced”. Thus, the term “plant-derived growth factor” refers in the context of the application generally to a heterologous growth factor, non-native to the host plant which is used as a production vehicle. The growth factor according to the present invention may be any human or non-human growth factor where its gene introduced into the plant or progenitors of the plant, preferably using recombinant technology. The isolated growth factor may be used as an active ingredient in a cosmetic composition or a therapeutic topical composition.

Methods for introducing and expressing foreign genes in plants are well known in the art. A plant that can be genetically transformed is a plant into which heterologous DNA sequence, including DNA sequence for a coding region, can be introduced, expressed, stably maintained, and transmitted to subsequent generations of progeny. Genetic manipulation and transformation methods have been used to produce barley plants that are using herbicide resistance including, for instance, bialaphos or basta, or antibiotic resistance, such as hygromycin resistance, as a selectable marker.

Suitable cultivars are selected and a suitable method for introduction of foreign gene selected. The term “transformation” or “genetic transformation” refers to the transfer of a nucleic acid molecule into the genome of a host organism, resulting in genetically stable inheritance. Host organisms containing the transformed nucleic acid fragments are referred to as “transgenic” organisms. A “transgenic plant host cell” of the invention contains at least one foreign, preferably two foreign nucleic acid molecule(s) stably integrated in the genome. Examples of methods of plant transformation include Agrobacterium-mediated transformation (De Blaere et al. 1987) and particle-bombardment or “gene gun” transformation technology (Klein et al. (1987); U.S. Pat. No. 4,945,050).

WO 2006/016381 describes a particular useful Barley cultivar amenable for transformation and describes in detail suitable transformation methods.

WO 2005/021762 discloses methods for modifying proteins by making chimeric proteins that are readily purified on a large scale.

Growth factors that are suitably produced and used according to the present invention may be selected from but are not limited to the species and groups including Transforming Growth Factors-b (or beta) (TGFs-b or TGFs-beta, including TGF beta1, TGF beta 2, TGF beta 3), Transforming Growth Factor-a (or alpha) (TGF-a or TGF alpha), TNF alpha, Epidermal Growth Factor (EGF), Platelet-Derived Growth Factors (PDGF AA, PDGF BB, PDGF Rb), Keratinocyte growth factor (KGF), Fibroblast Growth Factors a and b (aFGF and bFGF), FGF-4, FGF-6, Hepatocyte growth factor (HGF), Vascular Epithelial Growth Factor (VEGF) Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin-5 (IL-5), Interleukin-6 (IL-6), Interleukin-7 (IL-7), Interleukin-8 (IL-8), Interleukin-10 (IL-10), Interleukin 13 (IL-13), Interleukin-15 (IL-15), Interleukin-18 (IL-18), Interleukin-20 (IL-20), Leptin, Tumor Necrosis Factor-a (TNF-a), Tumor Necrosis Factor-b (TNF-b), Interferon-g (INF-g), Granulocyte Colony Stimulating Factor (G-CSFs), Granulocyte Macrophage Colony Stimulating Factor (GM-CSF), Macrophage Colony stimulating factor (M-CSF), Placenta Growth Factor (PLGF), Nerve Growth Factor (NGF), noggin, Bone morphogenesis Protein (BMP-4), and Thymosin beta 4.

In certain embodiments of the invention, the polypeptide of interest being produced in the transgenic plant contains an affinity tag at either N-terminal or C-terminal of the polypeptide, or at both ends. Such a tag may include repetitive HQ sequence, poly Histidine-tail, GST, CBM or any other useful affinity tag that simplifies purification of the heterologous peptide.

Hyaluronan is also called hyaluronic acid and hyaluronate, these terms are synonyms and interchangeable in the present context. Hyaluronan is an anionic, non-sulfated glycosaminoglycan distributed widely throughout connective, epithelial, and neural tissues.

The term skin care/dermatological composition as used herein encompasses both medical/pharmaceutical compositions for therapeutic dermatological applications as well as compositions for cosmetic use and compositions that can be used both for therapeutic and cosmetic use.

### Dosage

For topical therapeutic application in accordance with the invention, dose of growth factor is preferably in the range from 0.01 to 100 μg per gram of composition, and more preferably in the range 0,1 to 50 μg per gram. Local cosmetic compositions for the treatment of skin ageing or loss of hair preferably comprise from 0.2 to 50 ng of active substance per gram of composition.

The length of treatment varies depending on the pathology or on the desired effect. In the case of scleroderma treatment the application ranges from 1 day to 12 months according to the pathology severity. In the case of a treatment against natural or early ageing of the skin, the application ranges from 1 to 400 days, preferably for at least 30 days. Likewise, in the case of a treatment for preventing loss of hair or for promoting hair re-growth the application ranges from 1 to 400 days.

Dermatological compositions according to the invention can suitably be used for treatment of skin conditions including dry skin, eczema, dermatitis, rash, psoriasis, skin redness, and edema. Compositions of the invention are also useful for healing and reduction of scar tissue and healing and improving cracked skin on heels.

Preferably the transgenic plant extract is prepared from grains of barley containing any one or more of the proteins of the above listed growth factors, their mimetics or at least domains thereof that enable binding to, and activation of a growth factor receptor. The enclosed non-limiting examples show illustrative uses of different growth factors derived from transgenic barley extracts.

Extracts used according to the invention refer to protein extracts from the transgenic host plants, comprising the growth factor of interest. The growth factor can be only a minor component of the extract, provided that other proteins do not interfere with the activity of the growth factor or cause any other undesired effects. Such extracts are e.g. seed protein extracts from plants expressing the heterologous growth factor in their seeds. The extracts may also be purified to higher or lesser degree, i.e. they may be partially purified by one or more purification steps to enrich for the heterologous growth factor.

Numerous vehicles for topical application of cosmetic and pharmaceutical compositions are known in the art. See, e.g., Remington's Pharmaceutical Sciences, Gennaro, A. R., ed., 20th edition, 2000: Williams and Wilkins PA, USA. All compositions usually employed for topically administering cosmetic compositions may be used, e.g., creams, lotions, gels, dressings, shampoos, tinctures, pastes, serums, ointments, salves, powders, liquid or semiliquid formulation, patches, liposomal preparations, solutions, suspensions, liposome suspensions, W/O or O/W emulsions, pomades and pastes and the like as long as the heterologous protein as active ingredient is stabilized. Application of said compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used.

The compositions of the invention can be provided for parenteral, systemic or local use, comprising solutions, suspensions, liposome suspensions, W/O (water/oil) or O/W (oil/water) emulsions. In a preferred embodiment the active substance is formulated in a lyophilized form, mixed to suitable lyophilisation additives and ready to be redissolved with therapeutically acceptable diluents. Useful lyophilisation additives are: buffers, polysaccharides, sucrose, mannitol, inositol, polypeptides, amino acids and any other additive compatible with the active substance. In a preferred embodiment of the invention the active substance is dissolved in phosphate buffer (NaH₂PO₄/H₂O—Na₂HPO₄/2H₂O) in an amount such that the post-lyophilisation growth factor/phosphate ratio is comprised between 1:1 and 1:2. Diluents suitable for parenteral use are: water, physiological solutions, sugar solutions, hydroalcoholic solutions, oily diluents, polyols, like glycerol, ethylene or polypropylene glycol, or any other diluent compatible with the administration method as for sterility, pH, ionic strength and viscosity.

Preferably the vehicle of topical application is a formulation that is naturally anti-bacterial yet without any non-natural preservative or anti-microbial agent. It will be appreciated to use few ingredients and eliminate complex ingredients that may act as allergenics and/or irritants. The formulations should also ensure long term stability of the active protein ingredients, preferably providing long shelf life such as one year or longer at room temperature storage.

In a preferred embodiment the active compound, plant-produced recombinant growth factor of choice, is added to a formulation suitable for topical application containing one or more of glycerol, a salt such as but not limited to sodium chloride, potassium chloride and Calcium Chloride, where calcium chloride is the most preferred, purified water, and ethanol, and preferably all of those. Such compositions are surprisingly shown to effectively stabilize the recombinant protein represented by the growth factor of choice. It is an aspect of the present invention that this formulation effectively stabilizes recombinant proteins whether or not the proteins are glycosylated. The formulation is preferably antibacterial by nature and therefore particularly suitable as a topical formulation for dermatological and cosmetic use.

The composition of the invention may furthermore comprise an optional additive such as hyaluronic acid (hyalorunate).

In the case of emulsions or suspensions, the composition may contain suitable surfactants of non-ionic, zwitterionic, anionic or cathionic type commonly used in the formulation of medicaments. Oil/water (O/W) hydrophilic emulsions are preferable for parenteral systemic use, whereas water/oil (W/O) lipophilic emulsions are preferable for local or topic use.

Moreover, the compositions of the invention may contain optional additives like isotonic agents, such as sugars or polyalcohols, buffers, chelating agents, antioxidants, antibacterials.

Liquid forms according to the invention can comprise solutions or lotions. These may be aqueous, hydroalcoholic, like ethanol/water, or alcoholic and are obtained by solubilizing the lyophilized substance.

Alternatively, active substance solutions, may be formulated in form of gel by addition of known gelling agents, like: starch, glycerin, polyethylene or polypropylene glycol, poly(meth)acrylate, isopropyl alcohol, and hydroxystearate.

Other types of compositions for topic use are emulsions or suspensions in form of pomades, pastes, creams. W/O emulsions are preferable, providing a faster absorption. Examples of lipophilic excipients are: liquid paraffin, anhydrous lanolin, white vaseline, cetyl alcohol, stearyl alcohol, vegetable oils, mineral oils. Agents increasing cutaneous permeability, thereby facilitating the absorption, may advantageously be used. Examples of such agents are physiologically acceptable additives like polyvinyl alcohol, polyethylenglycol or dimethylsulfoxide (DMSO).

Other additives used in the topic compositions are isotonic agents, like sugars or polyalcohols, buffers, chelating agents, antioxidants, antibacterials, thickeners, dispersants.

It follows that the preparations may further contain conventional components usually employed in preparations described herein, including oils, fats, waxes, surfactants, humectants, thickening agents, antioxidants, viscosity stabilizers, chelating agents, buffers, preservatives, perfumes, dyestuffs, lower alkanols, and the like.

Delayed-release compositions for local or systemic use may be useful, and comprise polymers like polylactate, poly(meth)acrylate, polyvinylpyrrolidone, methylcellulose carboxymethylcellulose and other substances known in the art. Delayed-release compositions in form of subcutaneous implants based on, e.g. polylactate or other biodegradable polymers may be useful as well.

Though the active substance is preferably packaged in lyophilized and hence stable form, the pharmaceutical compositions advantageously comprise substances stabilizing the growth factor in the active form. Such stabilizers inhibit the formation of intermolecular disulfide bonds, thereby preventing the polymerization of the active substance. However, the amount of stabilizer should be carefully measured in order to concomitantly prevent the reduction of the active substance to the inactive monomeric form. Examples of such substances are: Cystein, Cysteamine, or glutathione in reduced form.

Non-limiting examples of oils include fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearite, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants include butylated hydroxytoluene, butylated hydroxyanisole, propyl gallate, citric acid and ethoxyquin; examples of chelating agents include disodium edetate and ethanehydroxy diphosphate; examples of buffers include citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. These substances are merely exemplary, and those of skill in the art will recognize that other substances may be substituted with no loss of functionality.

### EXAMPLES

### Example 1

### Preparation of Transgenic Plant Extract Containing Recombinant EGF

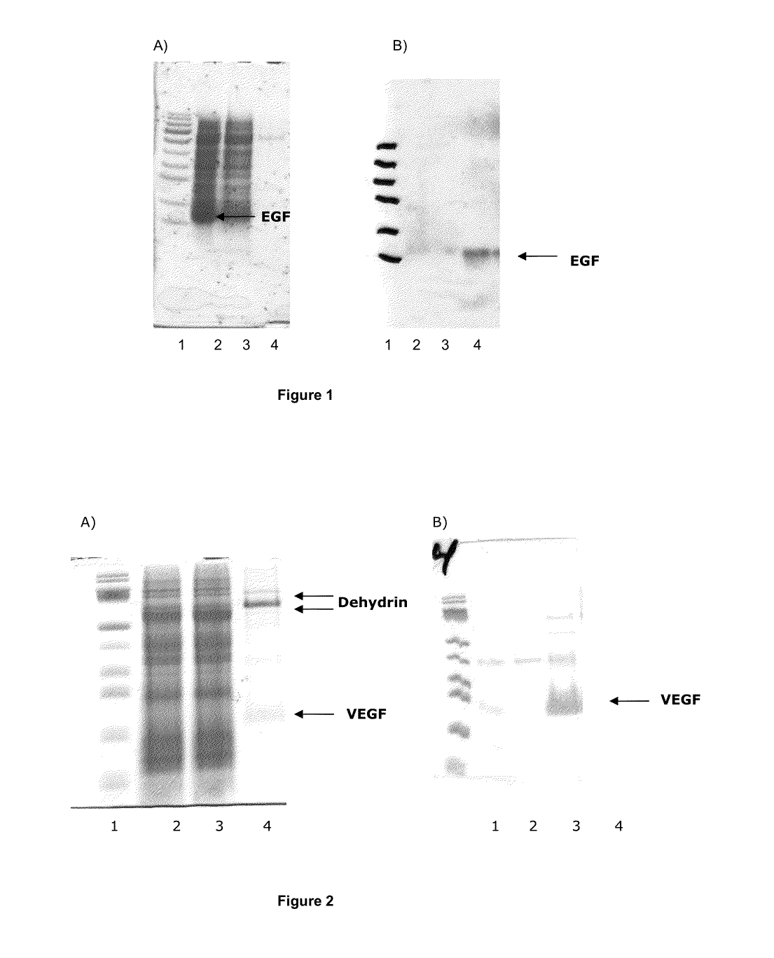

A transgenic plant extract was prepared by milling the harvested transgenic barley seeds containing a growth factor EGF, in a mill to obtain fine powder (flour). Extraction buffer added (50 mM potassium phosphate pH 7.0) to the milled barley flour in a volume/weight ratio of 5/1 of extraction buffer to milled flour. The resulting solution was stirred for 60 minutes at 4° C. Solids were separated from the liquid extract by centrifugal force, centrifuging at 8300 rpm in a refrigerated Centrifuge (Heraeus Primo R) or more, for 15 minutes, and the supernatant decanted off to a fresh vial. The growth factor content of the extract was analyzed by SDS-PAGE and Western blotting with a EGF specific antibody. In this experiment the EGF content was about 0.01% of the protein content of the extract. The results are shown in FIG. 1.

FIG. 1 (A) shows a Coomassie Blue stained part of gel staining the total soluble proteins. (B) shows the western blot with anti EGF antibody of the same extract showing the presence of the growth factor EGF in the transgenic plant extract. Lanes: 1: size marker, 2: transgenic barley seed extract, 3: flow-through from IMAC purification step, 4: elution of EGF from IMAC capture step.

### Example 2

### Partially Purified Transgenic Plant Extract Containing Growth Factor VEGF and Dehydrins

VEGF containing transgenic barley seed extract prepared according to Example 1 was processed further by adding to the extract an IMAC chromatography resin that effectively binds the VEGF. The mixture of extract and resin was stirred in 50 mM potassium phosphate, 0.5 M NaCl, 50 mM imidazole; pH7.0 at +4° C. for 60 minutes. The IMAC resin was separated from the liquid by centrifugation at 5000×g for 15 minutes. The liquid phase was decanted off and the resin was resuspended in washing buffer (50 mM potassium phosphate, 0.5 M NaCl, 50 mM imidazole; pH7.0) and spun down and the liquid phase decanted off the resin. The washing was repeated for 3 times. The resin was resuspended in elution buffer containing imidazole (50 mM potassium phosphate, 0.5 M NaCl, 500 mM imidazole; pH7.0) to elute the VEGF off the resin and after centrifugation the supernatant was decanted off the resin and run through gel filtration chromatography for buffer exchange. The resulting protein peak was analyzed on SDS-PAGE and Western blot. In this case the VEGF was present as approximately 25% of the protein extract. The results are shown in FIG. 2.

FIG. 2. Partially purified transgenic plant extract containing the recombinant growth factor VEGF and dehydrins. A) Coomassie blue stained SDS-PAGE gel staining total proteins present in the extract. B) Western blot showing the presence of VEGF in the partially purified barley seed extract containing dehydrin. Lane numbering: 1 Size standard, 2 Extract, 3 IMAC flowthrough, 4 IMAC elute. Arrows indicate the presence of dehydrins and VEGF in the partially purified extract, that have been identified with amino acid sequencing.

### Example 3

### Partially Purified Transgenic Plant Extract Containing Barley Globulin and IGF-1

A transgenic barley seed extracted in extraction buffer II (50 mM potassium phosphate pH7.0 200 mM NaCl) according to Example 1. The extract was bound to and eluted off IMAC resin as explained in Example 2, and subsequently the elute was desalted and the buffer changed to 100 mM KPi pH 6.8. The partially purified extract was then processed further by adding it to an ion exchange chromatography resin under conditions that effectively bind the IGF-1, in where the matrix used was SP-Sepharose (GE Healthcare). The corresponding fractions were analyzed with SDS-PAGE and silverstaining and western blot with specific anti IGF-1 antibodies. The IGF-1 content of the partially purified plant extract was 60%.

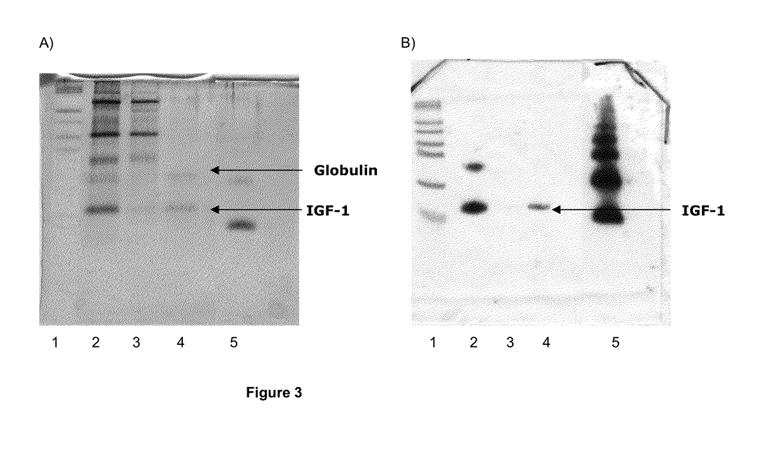

FIG. 3. Partially purified transgenic plant extract containing barley globulin and IGF-1 A) Silverstained SDS-PAGE gel. Lane numbering: 1 size standard, 2 desalted and buffer exchanged elute from IMAC column, 3 flow through from SP-Sepharose IEC, 4 elute from SP-Sepharose IEC containing the IGF-1 and barley globulin. 5 positive control: recombinant IGF-1 produced in bacteria.

### Example 4

### A Purified and Isolated Growth Factor IL-1 Alpha Purified from Transgenic Barley Seed Extract

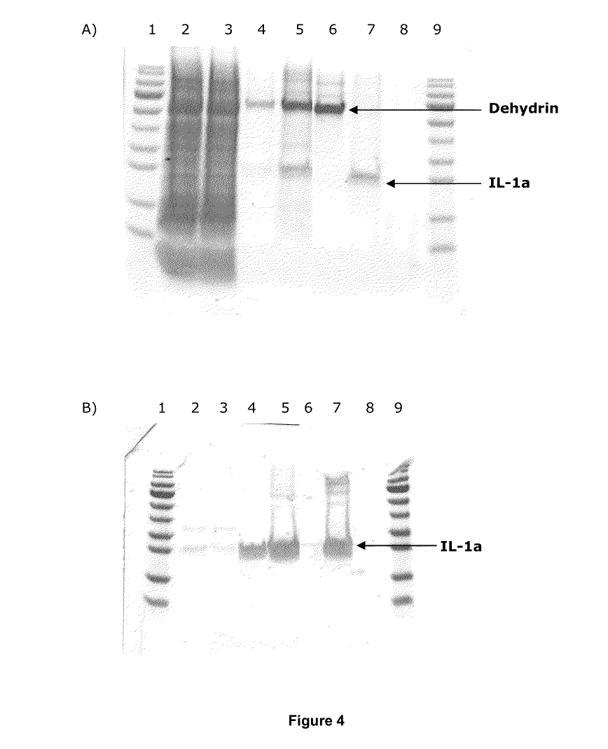

An extract of a transgenic plant may be purified further to isolate a growth factor, in a purified form: The IMAC elute is, after buffer exchange with gel filtration, applied to an ion exchange column Sepharose FF and the proteins in the extract were separated by stepwise elution increasing the NaCl content of the elution buffer. It was possible in this manner to successfully separate the growth factor from the dehydrin. As shown in FIG. 4 a growth factor can be purified to a high purity, >95% (lane 7) in this manner resulting in an isolated and purified IL-1 alpha isolated and purified from a transgenic plant extract.

FIG. 4 Purification and isolation of IL-1 alpha from transgenic plant extract. A) Coomassie blue stained SDS-PAGE gel staining total proteins present in the extract. B) Western blot of IL-1 alpha containing extracts. Lane numbering: 1 and 9 size markers, 2 extract, 3 IMAC Flow through, 4 IMAC elute, 5 concentrated IMAC elute, 6 35% NaCl elute from IEC, 7 75% NaCl elute, 8 100% NaCl elute. D: dehydrin, IL-1a: Interleukin 1 alpha.

### Example 5

### Use of a plant-derived growth factor in a composition

The following examples illustrate formulations of the cosmetic composition according to the present invention but are not intended to limit the invention in any way.

### Formulation 1: Skin Softener (Skin Lotion, Serum)

Stable compositions can be prepared buffered in the pH range of 6-9.

| Ingredients | Amounts (% by weight) |
|---|---|
| EGF (from transgenic plant) | 0.00025 |
| Glycerol | 10-90% |
| Calcium Chloride | 0.1 mM-200 mM |
| Purified water | q.s |
| Sodium hyaluronate | 0.01-2 |
| Ethanol | 0.1-10 |

### Formulation 2: Nutrient Emulsion (Milk Lotion)

| Ingredients | Amounts (% by weight) |
|---|---|
| EGF (from transgenic plant) | 0.0002 |
| Propyleneglycol | 6.0 |
| Glycerin | 4.0 |
| Triethanolamine | 1.2 |
| Tocopherylacetate | 3.0 |
| Liquid paraffin | 5.0 |
| Squalene | 3.0 |
| Makadamia nut oil | 2.0 |
| Polysorbate 60 | 1.5 |
| Sorbitan sesquioleate | 1.0 |
| Carboxyvinylpolymer | 1.0 |
| Flavor | 0.2 |
| Methylparaben | 0.2 |
| Imidazolidinyl Urea | 0.2 |
| Purified water | q.s. |

### Formulation 3: Nutrient Cream

| Ingredients | Amounts (% by weight) |
|---|---|
| VEGF (from transgenic plant) | 0.0005 |
| Vaseline | 7.0 |
| Liquid paraffin | 10.0 |
| Wax | 2.0 |
| Polysorbate 60 | 2.0 |
| Sorbitan sesquioleate | 2.5 |
| Squalene | 3.0 |
| Propyleneglycol | 6.0 |
| Glycerin | 4.0 |
| Triethanolamine | 0.5 |
| Carboxyvinylpolymer | 0.5 |
| Tocopherylacetate | 0.1 |
| Flavor | 0.2 |
| Methylparaben | 0.2 |
| Imidazolidinyl Urea | 0.2 |
| Purified water | q.s. |

### Formulation 4: Massage Cream

| Ingredients | Amounts (% by weight) |
|---|---|
| EGF (from transgenic plant) | 0.0002 |
| Propyleneglycol | 6.0 |
| Glycerin | 4.0 |
| Triethanolamine | 0.5 |
| Wax | 2.0 |
| Tocopherylacetate | 0.1 |
| Polysorbate 60 | 3.0 |
| Sorbitan sesquioleate | 2.5 |
| Cetearyl alcohol | 2.0 |
| Liquid paraffin | 30.0 |
| Carboxyvinylpolymer | 0.5 |
| Flavor | 0.2 |
| Methylparaben | 0.2 |
| Imidazolidinyl Urea | 0.2 |
| Purified water | q.s. |

### Formulation 5: Facial Pack

| Ingredients | Amounts (% by weight) |
|---|---|
| TGF-a (from transgenic plant) | 0.0005 |
| Propyleneglycol | 2.0 |
| Glycerin | 4.0 |
| Carboxyvinylpolymer | 0.3 |
| Ethanol | 7.0 |
| PEG-40 Hydrogenated Castor Oil | 0.8 |
| Triethanolamine | 0.3 |
| Flavor | 0.2 |
| Methylparaben | 0.2 |
| Imidazolidinyl Urea | 0.2 |
| Purified water | q.s. |

The formulations 1-5 can likewise be formulated with any alternative growth factor listed in the Detailed description.

### Formulation 6: W/O Emulsion for Topic Application.

An amount of lyophilized substance comprising 20 μg active substance is brought to 5 ml 10% ethanol hydro-alcoholic solution comprising 10% DMSO. The solution is emulsified in sterilized vegetable oil for cutaneous application using a surfactant suitable for W/O emulsions having a <10 HLB coefficient. The emulsion contains active substance equal to about 2 μg/g of composition.

### Formulation 7: O/W Emulsion

An amount of lyophilized substance comprising about 20 μg active substance is solubilized in 5 ml of hydro-alcoholic solution comprising 30% DMSO and emulsified with a suitable surfactant in a vegetable oil-based lipophilic solvent. The resulting O/W emulsion contains the active substance at a concentration of about 3 μg/g composition.

### Formulation 8: Topical Composition in Form of Gel.

An amount of lyophilized substance comprising 100 μg of active substance is brought in 20 ml 10% ethanol hydro-alcoholic solution comprising 20% DMSO. Then, the solution is additioned with a mixture of polyethylene glycol (400-4000) and polypropylene glycol. The active substance is present in an amount equal to 2 μg/g composition. The gel is suitable for cosmetic application.

### Formulation 9: A Topical Gel Formulation Containing Carbomer (1%)

| EGF (from transgenic plant) | 5 | mg |
|---|---|---|
| Carbomer 934P | 1 | g |
| Methyl paraoxybenzoate | 0.2 | g |
| Propylene glycol | 20 | g |
| Sodium hydroxide | q.s | |
| Distilled water for injection | q.s | |
| Total | 100 | g |

The formulation is prepared by using the above-mentioned components in given amounts according to a conventional method. Specifically, methyl paraoxybenzoate is dissolved in appropriate amounts of distilled water for injection, Carbomer 934P is added to the solution and dispersed therein with sting. The pH of the solution is controlled with sodium hydroxide, the solution is blended with propylene glycol and sterilized by heating. Then, filtered and sterilized solution of EGF in distilled water for injection is added thereto to obtain 100 g of formulation.

### Formulation 10: A Topical Formulation Containing Poloxamer (15%)

| EGF | 2.5 | mg |
|---|---|---|
| Poloxamer 407 | 15 | g |
| Methyl paraoxybenzoate | 0.2 | g |
| Sodium hydrogen phosphate | 272.18 | mg |
| Sodium chloride | 666.22 | mg |
| Phosphoric acid | q.s | |
| Propylene glycol | 20 | g |
| Distilled water for injection | q.s. | |
| Total | 100 | g |

The formulation is prepared by using the above-mentioned components in given amounts according to a conventional method. Specifically, phosphate buffer is prepared by using sodium hydrogen phosphate, sodium chloride and phosphoric acid in given amounts. Methyl paraoxybenzoate as the preservative is dissolved to the phosphate buffer. Poloxamer 407(BASF, Germany) is added to the solution and dispersed therein with string. Then the solution is blended with propylene glycol, dispersed therein with stirring. Then, the pH of the solution is controlled with sodium hydroxide, the solution is blended with propylene glycol and sterilized by heating. Then, filtered and sterilized solution of EGF in distilled water for injection is added thereto to obtain 100 g of formulation.

### Formulation 11: A Cream Formulation Containing Carbomer (0.1%)

| EGF | 0.05 | mg |
|---|---|---|
| Glycerin | 4.5 | g |
| Methyl paraoxybenzoate | 0.15 | g |
| Propyl paraoxybenzoate | 0.05 | g |
| Carbomer 940 | 0.1 | g |
| Steary alcohol | 1.75 | g |
| Cetyl alcohol | 4.00 | g |
| Span #60 | 0.50 | g |
| Polyoxyl #40 stearate | 2.00 | g |
| Triethanolamine | q.s | |
| Distilled water for injection | q.s | |
| Total | 100 | g |

The formulation is prepared by using the above-mentioned components in given amounts according to a conventional method. Specifically, glycerin and methyl paraoxybenzoate are dissolved in appropriate amounts of distilled water for injection, Carbomer 940(BF Goodrich, U.S.A.) is added to the solution and dispersed therein with stirring. Then, propyl paraoxybenzoate and the others are added to the solution and emulsified with melting. Then, the solution is sterilized after controlling pH with triethanolamine, and mixed with filtered and sterilized solution of EGF expressed and isolated from plant) in distilled water for injection to obtain 100 g of formulation.

### Example 6

### Stability Test of Plant-Produced EGF

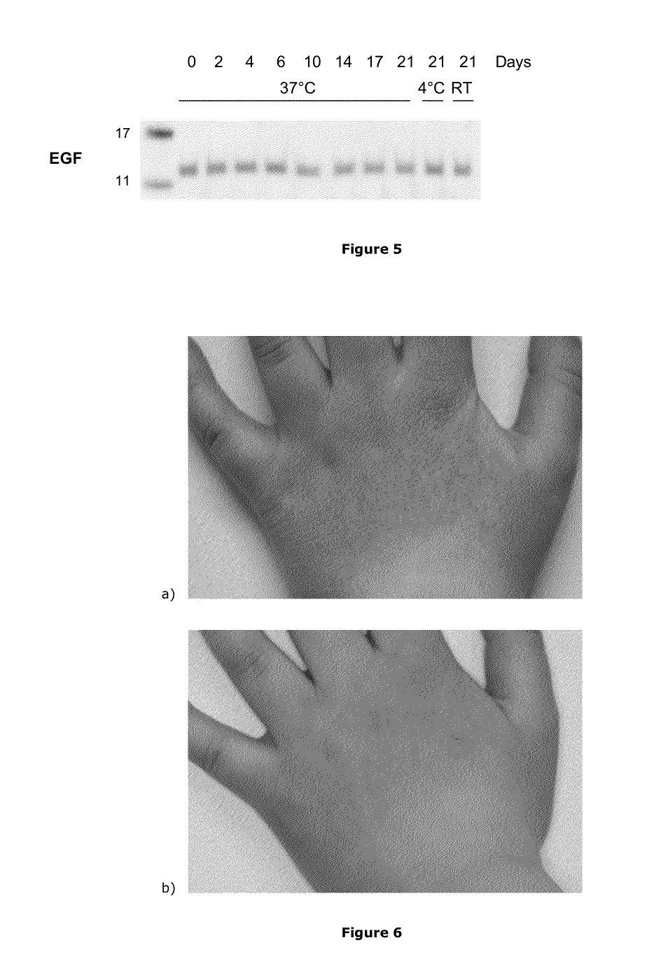

This Example shows a stability challenge test of a purified, reconstituted freeze-dried, non-glycosylated, plant-made EGF in a formulation as in Example 5, formulation 1, incubated at various temperatures; refrigerated at +4° C., incubated at +37° C. and room temperature (RT) for up to 3 weeks. Results are shown in FIG. 5. Size markers 11 and 17 kDa in the first lane. The results show excellent stability of the growth factor at 37° C., RT and at +4° C. for several weeks. According to descriptions by manufacturers of bacterially (E. coli) manufactured the reconstituted, purified form of recombinant EGF from bacteria is only stable for one week at 2° C.-4° C. (Ref.http://www.cellsciences.com/PDF/CRE100.pdf)

### Example 7

### Application Alleviating Winter Eczema on Hands

The composition of the invention containing partially purified transgenic barley seed-extract with plant-derived heterologous Epidermal Growth Factor (EGF) in a protein stabilizing formula of Formulation 1 of Example 5 is applied topically to dry-cracked, itchy, red skin with rash (i.e. winter eczema) of a 9-year old boy. Three drops of the topical formula are distributed evenly on the back of the hand clearly suffering from winter eczema. FIG. 6a) exhibits the hand before topical application of the composition of the invention as the formula, and FIG. 6b) exhibits the same hand 24 hours after the treatment with the composition of the invention, showing clear signs of skin restoration and alleviating the symptoms of the winter eczema.

### Example 8

### Winter Eczema

### “Winter Foot”

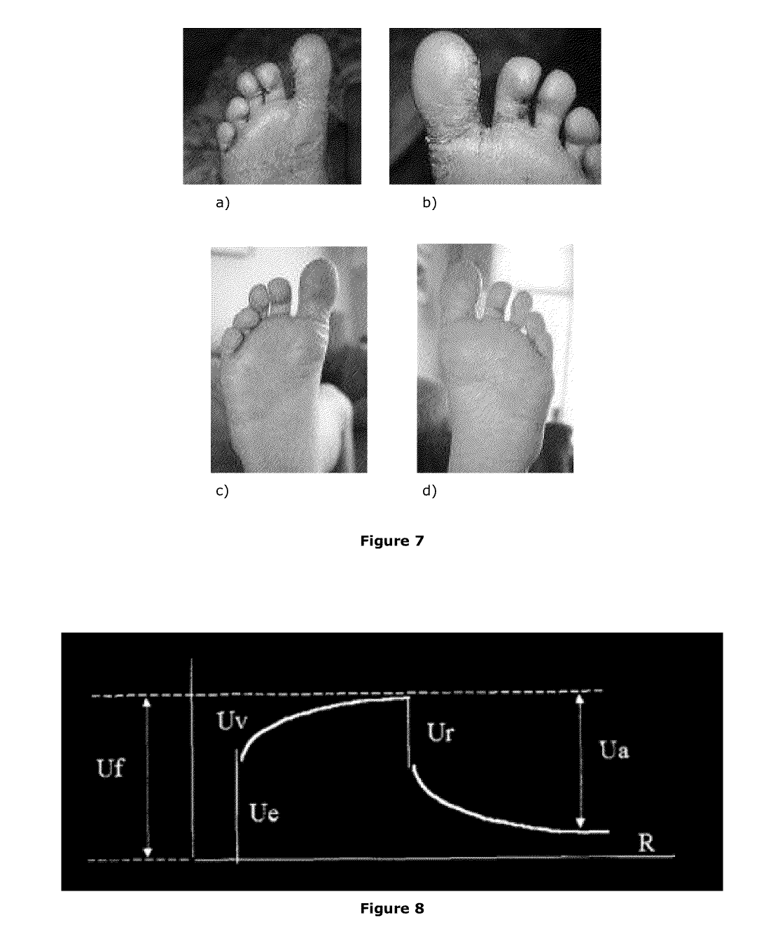

FIGS. 7 a) and b) exhibit the feet of a 10 year old boy with “winter-feets” resulting in severe skin problems. The dry, itchy, red skin causes irritation and bleeding from deep cracks of the skin and severly compromised epidermis.

Pictures 7 a) and b) taken before start of treatment and Pictures 7 c) and d) are taken after 5 days of daily topical application, 4-5 drops per foot, with the composition of the invention containing partially purified transgenic barley seed-extract with plant-derived heterologous Epidermal Growth Factor (EGF) in a protein stabilizing formula comprising Formulation 1 in Example 5. The epidermis has healed and has to great extent restored elasticity and softness and rehydration.

### Example 9

### Skin Elasticity

To objectively measure the effect of the composition of the invention containing partially purified transgenic barley seed-extract with plant-derived heterologous Epidermal Growth Factor (EGF) in a protein stabilizing formula comprising Formulation 1 in Example 5, on elasticity and firmness of facial skin the mechanical properties of skin were evaluated by means of controlled suction technology.

A typical skin deformation curve is illustrated in FIG. 8. The following parameters were analyzed: Ue, immediate distension—measures the ability of the skin to return to its initial position after deformation and is related to the function of elastic fibers.; Uv, delayed distension; [R0] Uf, final distension [skin distensibility]; Ur, immediate retraction; R, residual deformation at the end of measuring cycle [resilient distension]; [R2] Ua/Uf, gross-elasticity of the skin, including viscous deformation; [R5] Ur/Ue, neto-elasticity of the skin without viscous deformation; [R7] Ur/Uf, biological elasticity, i.e., the ratio of immediate retraction to total distension; [R6] Uv/Ue, the ratio of viscoelastic to elastic distension; and R8, viscopart, i.e., the area under the suction part of the deformation curve. The average values of two measurements were used in subsequent calculations.

Using non-invasive suction device (Cutometer® MPA580) the mechanical properties of facial skin was studied. This device measures the biomechanical properties of skin pulled into a small aperture and subjected to negative pressure. Two different skin-care formulations, Serum containing composition of the invention with the stabilized heterologous plant-produced EGF and corresponding Serum devoid of EGF, were applied each on opposite sides of the face including cheek, eye-contour and forehead. After four (4) months treatment elasticity and firmness were measured using Cutometer® MPA580. In brief, a progressive increase in suction from 0 to 450 mbar was applied to the skin, with a 2s application time of constant pressure, followed by a 2s relaxations period, with a total running time of 4 seconds. Skin elasticity and viscoelasticity was measured and evaluated on each side of the face, followed by value comparison between the two different treated areas.

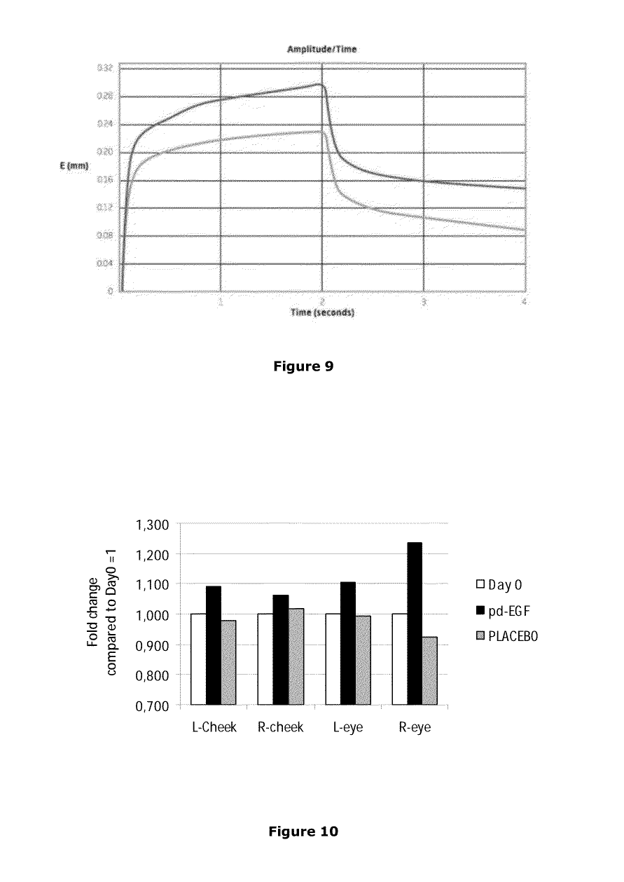

Results are shown in FIG. 9. Skin treated with EGF (upper curve) was characterized by significantly higher elastic parameters (i.e. Ue, Ur, Ua/Uf, Ur/Uf, Ur/Ue) and lower Viscoelasticity parameters (i.e. Uv and Uv/Ue). Apparently, when comparing the two differently treated areas, the increase in skin elasticity and decrease in viscoelasticity correlates with treatment with Serum containing composition of the invention with the stabilized heterologous plant-derived EGF. Skin treated with the composition of the current innovation was characterized by increased skin elasticity and firmness.

### Example 10

### Evaluation of Facial Skin Elasticity Following One Month Application of Formulated Plant Extract Containing Plant-Derived Heterologous EGF (Pd-EGF)

Subjects were 12 females between 30 and 70 years. This study did not include any inclusion criteria of obvious signs of aging skin. Eight (8) of the subjects used transgenic barley seed-extract with plant-derived heterologous Epidermal Growth Factor (EGF) in a protein stabilizing formula comprising Formulation 1 in Example 5, while four (4) subjects got serum without transgenic barley seed extract i.e. placebo serum, to apply on forehead, cheeks and eye contour twice daily for one (1) month. Subjects were allowed to continue their normal daily skin care procedures during the study-period.

At the beginning (baseline=Day 0) and at the end of the application period non-invasive skin analysis investigation was performed on the skin surface of the treated areas and results from baseline (Day 0) compared to the results after one month application. Quantitative measurements were obtained by Soft Plus Skin Analyzing System (Callegari1930). Parameter tested in this study was skin elasticity as measured by resistance to suction. All data is analyzed as the average values before (baseline=Day0) and average values at the end of the study for all individuals completing the study. The final results are presented (FIG. 10) as the fold-induction (i.e. relative value 1=Day 0) of treated compared to untreated areas compared to the baseline values.

### Example 10

### Antimicrobial Activity

The Example shows test of microbial challenge of a composition of the invention which is in accordance with Example 5, formulation 1, and the determination of antimicrobial preservation efficacy.

The composition was challenged with bacteria to establish antibacterial properties of the composition. 0.5 ml of liquid culture of Pseudomonas aeruginosa (strain ATCC 9027) was seeded onto 50 ml of stabilizing composition. Samples were incubated under standardized conditions and the number of bacteria determined based on methods described in detail for “Efficacy of Antimicrobial preservation” in European Pharmacopoeia 5.1.3.; Inoculum is prepared from stock culture of the bacteria, sterile suspension fluid containing 9 g/L of Sodium chloride. The culture is diluted with the fluid to obtain 107 bacteria per ml and 1 ml is added as inoculum in the container containing the stabilizing composition and mixed thoroughly. The inoculated product is maintained at 22° C. protected from light. Samples of 1 ml are drawn from the inoculated product at specified time intervals and the number of bacteria determined by plate count.

| Time from seeding | Number (log10) of P. aeruginosa |
|---|---|
| (days) | per ml of stabilizing composition |
| 0 | 7.40 |
| 2 | 4.43 |
| 7 | 0.48 |
| 14 | 0 |
| 28 | 0 |

The results show clear antimicrobial activity of a composition of the present invention and fulfill the recommended efficacy of antibacterial activity according to European Pharmacopoeia for Topical preparations, without the use of conventional preservatives and antibacterial agents. Thus, these formulations are suitable for topical cosmetic and/or therapeutic compositions and circumvent possible side effects of potentially irritating preservatives, antimicrobial agents, and other additives which are compromising for sensitive skin.

### REFERENCES

Ito I, Fixman E D, Asai K, Yoshida M, Gounni A S, Martin J G, Hamid Q. “Platelet-derived growth factor and transforming growth factor-beta modulate the expression of matrix metalloproteinases and migratory function of human airway smooth muscle cells.” Clin Exp Allergy. 2009 September; 39(9):1370-80. Epub 2009 Jun. 11.
Nakatani Y, Tanimoto K, Tanaka N, Tanne Y, Kamiya T, Kunimatsu R, Tanaka E, Tanne K. “Effects of hyaluronan oligosaccharide on the expression of MMP-1 in periodontal ligament cells.” Arch Oral Biol. 2009 August; 54(8):757-63. Epub 2009 Jun. 11.
Technical specification sheet, EGF Recombinant Human Epidermal Growth Factor, Cell Sciences, MA, USA (http://www.cellsciences.com/PDF/CRE100.pdf)
   5.1.3 Efficacy of Antimicrobial Preservation,: 528-529, European Pharmacopoeia 6.0

1. A skin care/dermatological composition comprising hyaluronic acid and at least one growth factor derived from a transgenic plant and at least one pharmaceutically and/or cosmetically acceptable excipient.

2. The skin care/dermatological composition of claim 1 comprising an excipient formula that stabilizes the growth factor protein.

3. The skin care/dermatological composition of claim 1 wherein the hyaluronic acid is provided in a concentration in the range of about 0,01 to about 2% by weight.

4. The skin care/dermatological composition of claim 1 wherein the growth factor is provided as a component of a transgenic plant extract comprised in the cosmetic composition.

5. The skin care/dermatological composition of claim 4 where the growth factor is present in the transgenic plant extract in amount in the range of about 0.0001% to about 70% of the total protein content.

6. The skin care/dermatological composition of claim 4 wherein said plant extract has been purified to enrich for said growth factor.

7. The skin care/dermatological composition of claim 4 suitable for treatment of a skin condition selected from one or more of dry skin, eczema, dermatitis, cracked skin, rash, skin redness, scar tissue, psoriasis, and edema.

8. The skin care/dermatological composition of claim 2 wherein said protein stabilizing formula comprises glycerol, water, and calcium chloride.

9. The skin care/dermatological composition of claim 8 wherein said protein stabilizing formula comprises the following ingredients (% by weight): glycerol in the range from 10 to 90%, calcium chloride in the range from 0,1 mM to 200 mM, buffered in the pH range of 6-9 and purified water q.s.

10. The skin care/dermatological composition of claim 8, which does not contain a further antimicrobial agent or preservative.

11. The skin care/dermatological composition of claim 1 comprising more than one growth factor derived from transgenic plants.

12. The skin care/dermatological composition of claim 11 where said more than one growth factor are present as components of a mixture of extracts from transgenic plants.

13. The skin care/dermatological composition of claim 11 where said more than one growth factor are present in the mixture of transgenic plant extracts in an amount in the range of about 0.0001% to about 70% of the protein content.

14. The skin care/dermatological composition of claim 1 wherein the at least one growth factor or more than one growth factors is isolated and purified from transgenic plants to a level of purity in the range of about 70% to about 99.9%.

15. The skin care/dermatological composition of claim 1 wherein one or more plant-derived purified growth factors are added to a transgenic plant extract already containing a plant-derived growth factor.

16. The skin care/dermatological composition of claim 1 wherein the at least one growth factor or more than one growth factor is selected from the group consisting of Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), Placenta Growth Factor (PLGF), Nerve Growth Factor (NGF), Keratinocyte Growth Factor (KGF), Bone morphogenesis Protein (BMP-4), Hepatocyte Growth Factor (HGF), Leptin, Noggin, and Thymosin beta 4.

17. The skin care/dermatological composition of claim 1 wherein said growth factor or cytokine originates from corresponding human gene sequence.

18. The skin care/dermatological composition of claim 1 wherein said growth factor or cytokine originates from synthesized gene corresponding to human gene sequence for respective growth factor or cytokine.

19. The skin care/dermatological composition of claim wherein the transgenic plant extract contains dehydrins and/or globulins or other seed proteins.

20. The skin care/dermatological composition of claim 1 wherein the composition is in the form selected from creams, lotions, gels, dressings, shampoos, tinctures, pastes, ointments, salves, powders, liquid or semiliquid formulations, serums, patches, liposomal preparations, solutions, suspensions, liposome suspensions, W/O or O/W emulsions, ointments, pomades and pastes and a skin softener cream, a facial pack, a massage cream, and a nutrient cream or a nutrient emulsion.

21. A skin care/dermatological composition comprising at least one growth factor derived from a transgenic plant and a protein stabilizing formula comprising glycerol, and purified water q.s.

22. The skin care/dermatological composition of claim 21 further comprising a salt selected from sodium chloride, potassium chloride and calcium chloride.

23. The skin care/dermatological composition of claim 21, further comprising hyaluronic acid.

24. The skin care/dermatological composition of claim 22, comprising a growth factor derived from a transgenic plant and a protein stabilizing formula comprising at least the following ingredients (% by weight): glycerol in the range from 10 to 90%, calcium chloride in the range from 0,1 mM to 200 mM, buffering agent and purified water q.s.

25. The skin care/dermatological composition of claim 21 which does not contain a further antimicrobial agent or preservative.

26. The skin care/dermatological composition of claim 21 wherein said growth factor is one or more selected from the group consisting of Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), Placenta Growth Factor (PLGF), Nerve Growth Factor (NGF), Keratinocyte Growth Factor (KGF), Bone morphogenesis Protein (BMP-4), Hepatocyte Growth Factor (HGF), Leptin, Noggin, and Thymosin beta 4.

27-30. (canceled)

31. A method of manufacturing a topical product comprising mixing together a hyaluronic acid, at least one excipient and transgenic plant extract comprising a growth factor selected from Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), Placenta Growth Factor (PLGF), Nerve Growth Factor (NGF), Keratinocyte Growth Factor (KGF), Bone morphogenesis Protein (BMP-4), Hepatocyte Growth Factor (HGF), Leptin, Noggin, and Thymosin beta 4.

32. The method of claim 31 wherein said at least one excipient comprises glycerol, calcium chloride and water.

33. The method of claim 31 wherein said transgenic plant extract is barley seed extract.

34. The method of claim 31 further comprising isolating said growth factor from said transgenic plant extract.

35. (canceled)
